(19) United States
(12) Patent Application Publication
(10) Pub. No.: US 2008/0262025 A1
(43) Pub. Date: Oct. 23, 2008

(54) Processes for the Preparation of Zolpidem and its Hemitartrate

(76) Inventors: Yatendra Kumar, Haryana (IN); Prasad Mohan, Haryana (IN); Asok Nath, Haryana (IN); Tippasandra Chandrashekar, Haryana (IN); Rita Santhakumar, Haryana (IN); Somenath Ganguly, Haryana (IN)

Correspondence Address: RANBAXY INC., 600 COLLEGE ROAD EAST, SUITE 2100, PRINCETON, NJ, 08540, US

(21) Appl. No.: 11/572,017

(22) PCT Filed: Jul. 15, 2005
(86) PCT No.: PCT/IB2005/002043

§ 371 Date: May 2, 2008

(30) Foreign Application Priority Data
Jul. 16, 2004 (IN) 1313/DEL/2004
Aug. 19, 2004 (IN) 1549/DEL 2004

(51) Int. Cl. A61K 31/437; C07D 471/04; A61P 25/22; A61P 25/00

(52) U.S. Cl. 514/300; 546/121

(57) ABSTRACT

The invention relates to the preparation of a non-hygroscopic polymorphic form of zolpidem hemitartrate, designated as Form I, and pharmaceutical compositions including it. The invention also relates to use of the compositions for treating anxiety, sleep disorders and convulsions. The invention also relates to a process for the preparation of zolpidem or pharmaceutically acceptable salts thereof by condensing 3-bromo-N,N-dimethyl-4-oxo-4-p-tolyl-butyramide with 2-amino-5-methylpyridine in a polar aprotic solvent.

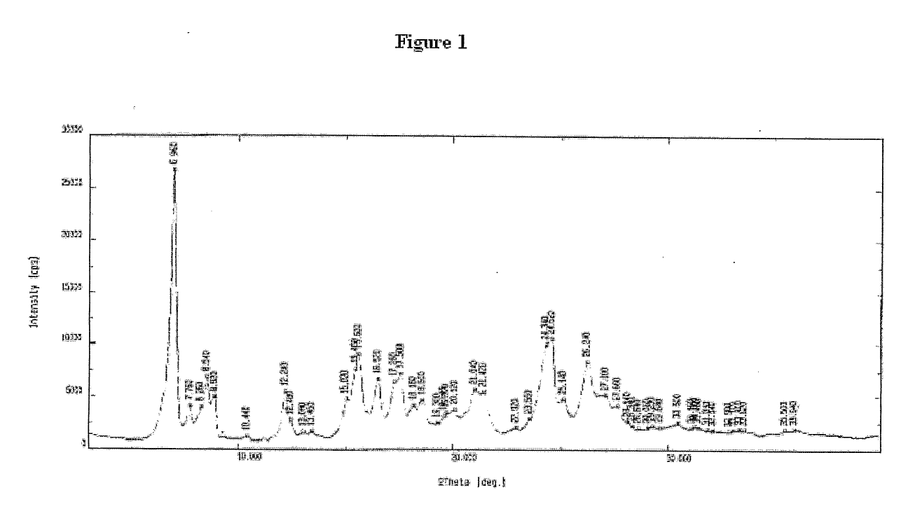

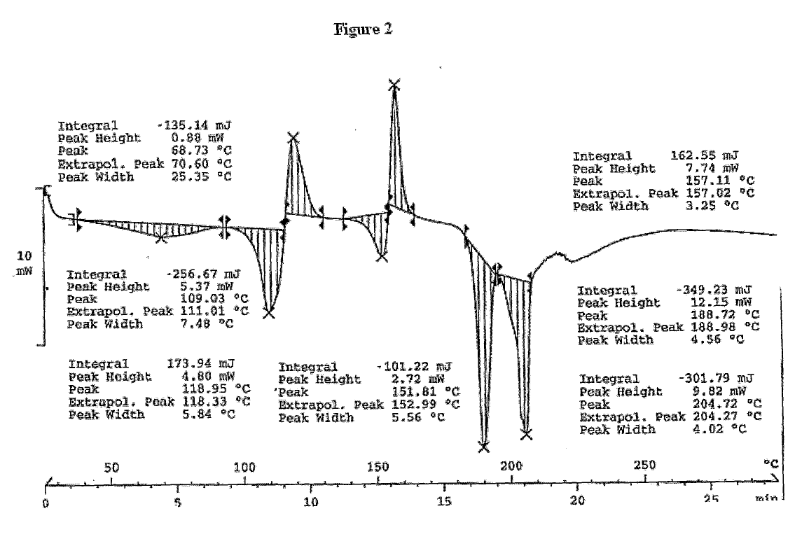

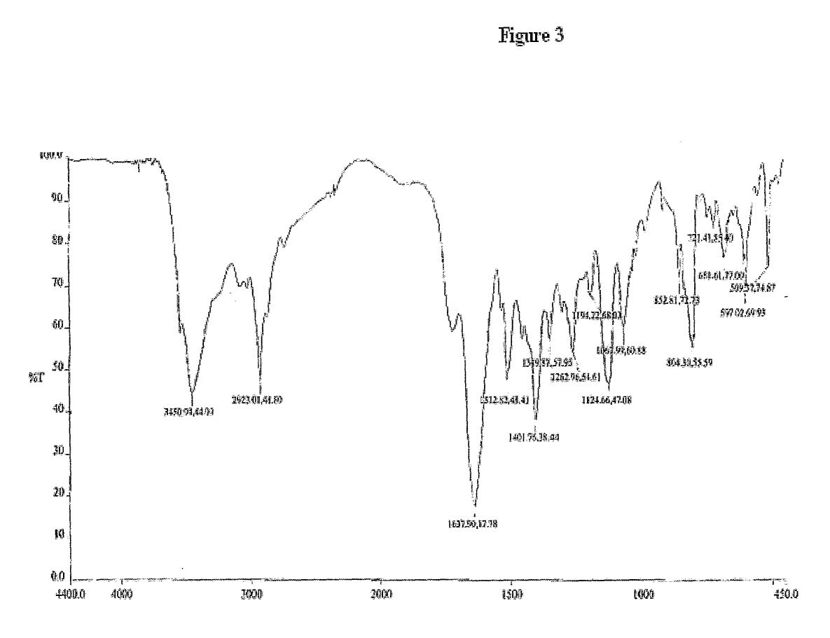

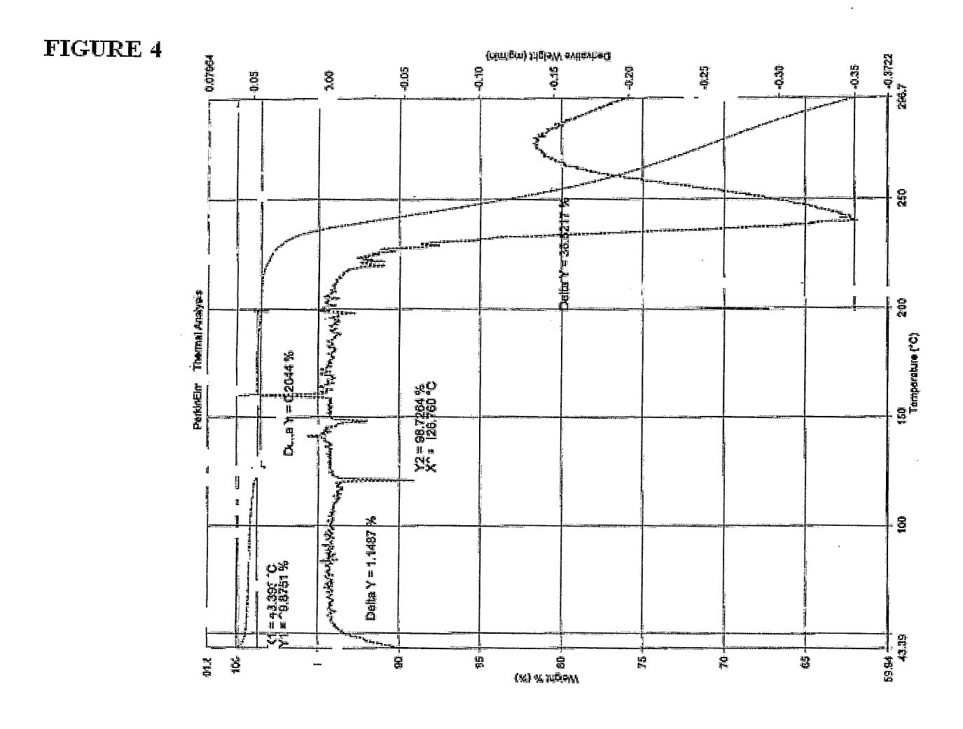

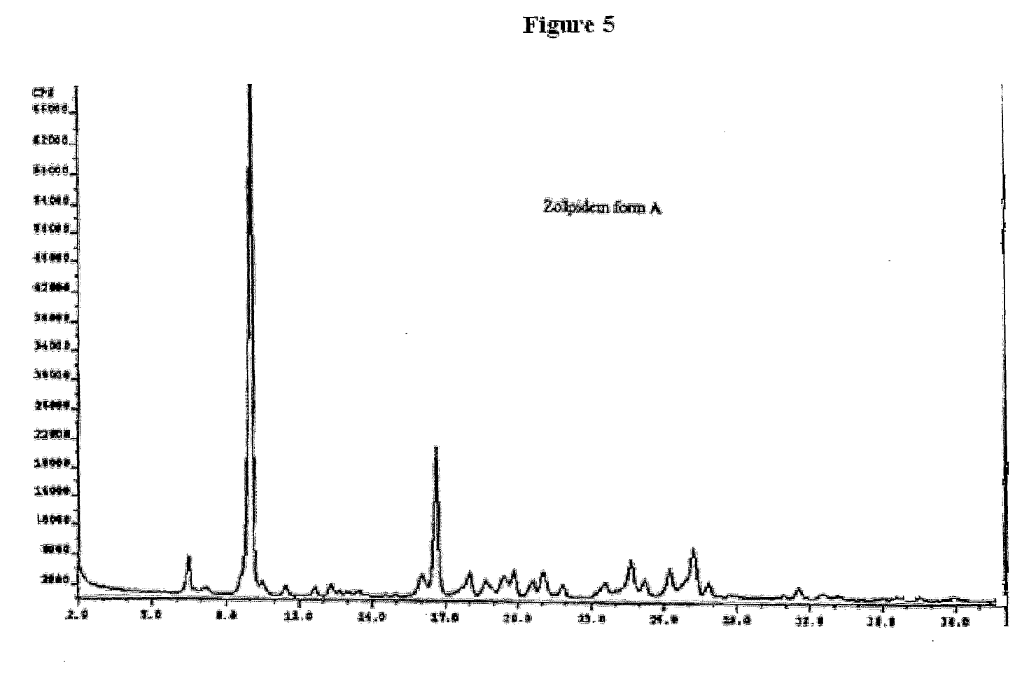

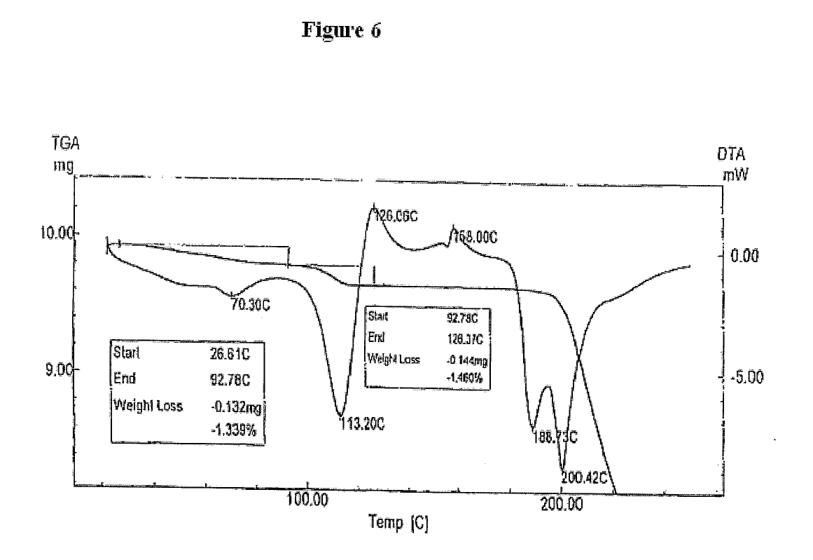

### FIELD OF THE INVENTION

The field of the invention relates to processes for the preparation of a polymorph of zolpidem hemitartrate. More particularly, it relates to the preparation of a non-hygroscopic polymorphic form of zolpidem hemitartrate and pharmaceutical compositions that include the non-hygroscopic polymorphic form, designated as Form I of zolpidem hemitartrate. The invention also relates to use of the compositions for treating anxiety, sleep disorders and convulsions. The field of the invention also relates to a process for the preparation of zolpidem or pharmaceutically acceptable salts thereof.

### BACKGROUND OF THE INVENTION

Chemically, zolpidem is N,N,6-trimethyl-2-(4-methylphenyl)-imidazo[1,2-a]pyridine-3-acetamide of Formula I. It is useful in the treatment of anxiety, sleep disorders and convulsions.

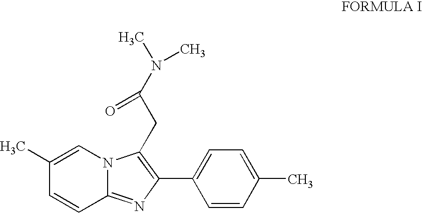

Zolpidem is commercially available as its hemitartrate salt of Formula II,

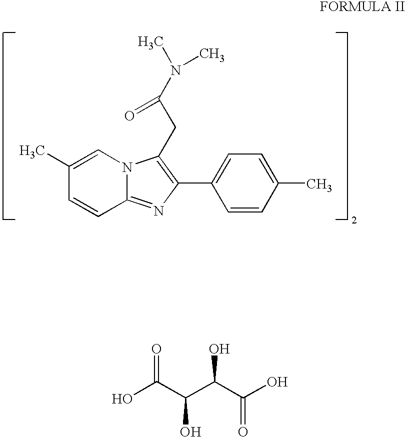

having 2:1 ratio of zolpidem base to L-(+)-tartaric acid.

The pharmacological profile of zolpidem is characterized by a strong hypnotic effect, together with weak anticonvulsant and muscle-relaxant properties, showing selectivity for benzodiazepine receptors with the biochemical characteristics and regional distribution of the benzodiazepine one subtype. While zolpidem is a hypnotic agent with a chemical structure unrelated to benzodiazepines, barbiturates, or other drugs with known hypnotic properties, it interacts with gamma-aminobutyric acid (GABA)-benzodiazepine receptor complex and shares some of the pharmacological properties of the benzodiazepines. The selective binding of zolpidem on the omega-1 receptor may explain the relative absence of myorelaxant and anticonvulsant effects in animal studies. Zolpidem shows both high affinity and selectivity toward non-benzodiazepine-2 receptors which results in improved activity and/or fewer side effects for the treatment of anxiety, sleep disorders and convulsions.

A general method for the synthesis of 2-phenylimidazo[1,2-a]pyridine is reported in J. Med. Chem., 40, 3109-3118 (1997). The preparation of zolpidem is, however, not discussed in this article. The reaction conditions employed therein are stringent and require higher temperatures.

Several processes have been reported for the preparation of zolpidem or its salt for example, in U.S. Pat. Nos. 4,382,938; 4,794,185; 4,356,283; 4,460,592; 4,501,745; 4,675,323; 4,808,594; 4,847,263; 6,281,360; 6,407,246; and 6,384,226; and International (PCT) Publication Nos. WO 01/38327 and WO 02/14316.

U.S. Pat. No. 4,382,938 discloses a process for the preparation of zolpidem base by treating zolpidic acid with dimethylamine in the presence of carbonyldiimidazole and tetrahydrofuran, followed by treating the mass with base and recrystallizing from ethanol to get zolpidem base. The product is not obtained with a high purity profile, thus making the process commercially difficult to implement.

U.S. Pat. No. 4,794,185 discloses a method for the preparation of zolpidem hemitartrate. The method involves treating zolpidem base with L-(+)-tartaric acid in methanol and allowing the hemitatrate salt to crystallize. The product obtained has a melting point of 197° C. (hereinafter designated as Form A of zolpidem hemitartrate).

Form A has some characteristic physico-chemical properties. It is very hygroscopic under normal storage conditions and can absorb up to about 5% w/w of moisture after exposure to atmospheric conditions. It is therefore difficult for a formulation scientist to prepare a pharmaceutical composition of Form A because the absorption of water results in problems of weight variation and content uniformity in the formulation.

Form A is further characterized by X-Ray diffraction (XRD) pattern. A typical XRD of Form A shows characteristic absorption peaks at two-theta values of 6.5, 9.0, 16.1, 16.6, 24.6 and 27.3. After micronization, the Form A exhibits some additional characteristic peaks than those mentioned above at two-theta values of 6.7, 8.6, 11.2, 15.4 and 17.3.

Form A exhibits a characteristic Differential Scanning Calorimetry (DSC) profile. Four endothermic peaks are observed at 70.3° C., 113.2° C., 188.73° C. and 200.42° C. and two exothermic peaks are obtained at 126.06° C. and 168.00° C.

The Thermal Gravimetric Analysis (TGA) and Karl Fischer Analysis of Form A shows that initially Form A has about 1.0% w/w of moisture, which after absorption of water rises up to 5%. The Karl Fischer analysis suggests that Form A can have up to 3% w/w to 5% w/w of moisture. TGA analysis supports this data. The endotherm at 110° C. in TGA analysis suggests partial desorption of water with an overall water content of about 3% w/w to 5% w/w.

U.S. Patent Application No. 20020077332 discloses various polymorphic forms of zolpidem hemitartrate, for example, anhydrous, hydrated, or solvated forms having specific X-Ray diffraction patterns, TGA profile and moisture or solvent content. These polymorphic forms are designated as anhydrous, monohydrate, dihydrate, trihydrate and tetrahydrate of zolpidem hemitartrate along with Forms B, C, D, E, F, G, H and L. With the stricter regulatory norms of solvent content and water content (e.g., European Pharmacopoeia specifies a water content of not more than 3% for zolpidem hemitartrate), it is very difficult to formulate the solvated and hydrated polymorphic forms disclosed in this application.

International (PCT) Patent Application No. PCT/IB01/01558 relates to processes for the preparation of N,N-dimethyl-3-(4-methyl)benzoyl propionamide of Formula III, which is a key intermediate in the synthesis of zolpidem.

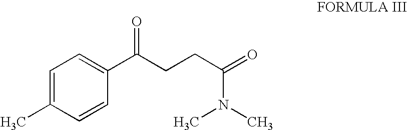

International (PCT) Patent Application No. PCT/IB32004/00245 discloses a process for the preparation of zolpidem hemitartrate by treating N,N-dimethyl-3-(4-methyl)benzoyl propionamide of Formula III with bromine to get bromo amide of Formula IV,

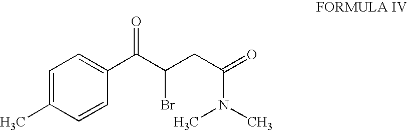

followed by treatment of the bromo amide of Formula IV with 2-amino-5-methylpyridine to obtain zolpidem base of Formula I, which is then converted to zolpidem hemitartrate of Formula II.

The present inventors have surprisingly found that zolpidem or a salt thereof can be prepared in high yield and purity by condensing bromo amide of Formula IV with 2-amino-5-methylpyridine in the presence of an organic solvent at a temperature of above 80° C. The inventors have also found that a novel polymorph of zolpidem hemitartrate can be obtained which is stable and non-hygroscopic when studied under extensive stability studies.

Thus, the present invention provides a process which does not result in a hygroscopic form; rather, a pure stable non-hygroscopic form is obtained. The non-hygroscopic zolpidem hemitartrate when made by the process of the present invention is easy to isolate and formulate thus making the process amenable for commercial scale use.

### SUMMARY OF THE INVENTION

In one general aspect there is provided a process for the preparation of zolpidem of Formula I,

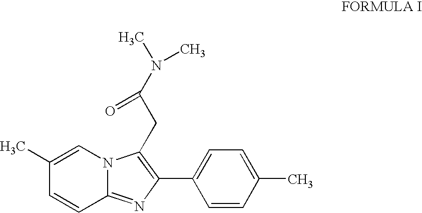

or a pharmaceutically acceptable salt thereof. The process steps include
a) condensing bromo amide of Formula IV,

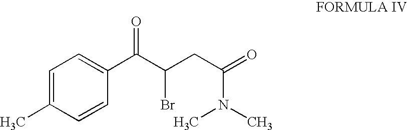

with 2-amino-5-methylpyridine in one or more polar aprotic solvents, at a temperature above 80° C.; and
b) isolating zolpidem of Formula I or a pharmaceutically acceptable salt thereof, by removal of the solvent.

The polar aprotic solvent may include one or more of methyl isobutyl ketone, ethyl methyl ketone, diisobutyl ketone, 1,4-dioxane, sulpholane, N,N-dimethylformamide, N,N-dimethylacetamide, acetonitrile, and N-methylpyrrolidone. Mixtures of all of these solvents are also contemplated.

Removing the solvent may include, for example, one or more of distillation, distillation under vacuum, evaporation, filtration, filtration under vacuum, decantation and centrifugation.

The product obtained may be further or additionally dried to achieve the desired moisture values. For example, the product may be further or additionally dried in a tray drier, dried under vacuum and/or in a Fluid Bed Drier.

The process may include further forming the product so obtained into a finished dosage form.

In one general aspect, the condensation of the bromo amide of Formula IV with 2-amino-5-methylpyridine is carried out in a polar aprotic solvent at a temperature from about 80° C. to about 120° C., for example from about 80° C. to about 100° C. It may be heated from about 10 minutes to about 24 hours.

In another general aspect, the reaction mixture containing zolpidem may be cooled before filtration to obtain better yields of the zolpidem or a salt thereof.

In another general aspect there is provided a non-hygroscopic polymorphic form of zolpidem hemitartrate (hereinafter designated as Form I).

The polymorphic form of zolpidem hemitartrate may have, for example, the X-ray powder diffraction pattern of FIG. I, the differential scanning calorimetry profile of FIG. II, and infrared spectrum of FIG. III.

In another general aspect there is provided a process for preparing polymorphic Form I of zolpidem hemitartrate. The process includes obtaining a solution of zolpidem base in one or more solvents; contacting the solution with L-(+)-tartaric acid to form a reaction mixture; adding an anti-solvent to the reaction mixture; and isolating Form I of zolpidem hemitartrate from the mixture thereof.

The process may include further drying of the product obtained.

In another general aspect there is provided a pharmaceutical composition that includes a therapeutically effective amount of the non-hygroscopic polymorphic Form I of zolpidem hemitartrate and one or more pharmaceutically acceptable carriers, excipients or diluents.

In another general aspect there is provided a method of treating anxiety, insomnia, sleep disorders, and convulsions in a warm-blooded animal, the method including providing to the warm-blooded animal a pharmaceutical composition that includes Form I of zolpidem hemitartrate.

The details of one or more embodiments of the inventions are set forth in the description below. Other features, objects and advantages of the inventions will be apparent from the description and claims.

### DESCRIPTION OF THE DRAWINGS

FIG. I is an X-ray powder diffraction pattern of Form I of zolpidem hemitartrate.

FIG. II is a differential scanning calorimetry thermogram of Form I of zolpidem hemitartrate.

FIG. III is an infrared spectrum of Form I of zolpidem hemitartrate.

FIG. IV is a thermogravimetry curve of Form I of zolpidem hemitartrate.

FIG. V is an X-ray powder diffraction pattern of Form A of zolpidem hemitartrate.

FIG. VI is a differential scanning calorimetry thermogram of Form A of zolpidem hemitartrate.

### DETAILED DESCRIPTION OF THE INVENTION

The inventors have developed a process for preparing zolpidem or its pharmaceutically acceptable salts. The process provides benefits with respect to economics and convenience to operate at a commercial scale.

The process steps include:
(a) condensing bromo amide of Formula IV,

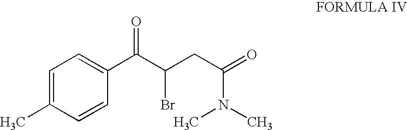

with 2-amino-5-methylpyridine in one or more polar aprotic solvents, at a temperature above 80° C.; and
(b) isolating zolpidem of Formula I or a pharmaceutically acceptable salt thereof, by removal of the solvent.

The starting material, the bromo amide of Formula IV, can be prepared as per the process disclosed in International (PCT) Patent Application No. PCT/IB2004/00245, the disclosure of which is incorporated herein by reference. The bromo amide intermediate of Formula IV is treated with 2-amino-5-methylpyridine in the presence of a suitable solvent at a temperature above 80° C. to get zolpidem base. Zolpidem base may then be converted to a pharmaceutically acceptable salt thereof by treating it with a suitable pharmaceutically acceptable acid. In particular, it may be treated with L-(+)-tartaric acid to get zolpidem hemitartrate.

Suitable solvents include polar aprotic solvents. The polar aprotic solvent may include one or more of methyl isobutyl ketone, ethyl methyl ketone, diisobutyl ketone, 1,4-dioxane, sulpholane, N,N-dimethylformamide, N,N-dimethylacetamide, acetonitrile, and N-methylpyrrolidone. Mixtures of all of these solvents are also contemplated. The reaction can be carried out a temperature range of from about 80° C. to about 120° C. in particular, it may be carried out at a temperature from about 80° C. to about 100° C.

The inventors have found a novel non-hygroscopic polymorphic form (designated Form I) of zolpidem hemitartrate. The polymorphic Form I is characterized by its X-ray diffraction pattern as shown in FIG. I, differential scanning calorimetry profile as shown in FIG. II, infrared spectrum as shown in FIG. III, and the thermogravimetry curve as shown in FIG. IV.

In general, the Form I of zolpidem hemitartrate may be characterized by X-ray diffraction peaks at about 7.0, 7.8, 8.6, 8.9, 12.2, 15.6, 16.5, 17.3, 24.3 and 26.0 degrees two-theta values.

Further, in general, Form I of zolpidem hemitartrate may be characterized by DSC endothermic peaks at about 70° C., 109° C., 189° C. and 204° C. It may be further characterized by exothermic peaks at about 119° C. and 157° C.

Further, Form I of zolpidem hemitartrate may be characterized by weight loss from about 1.0% up to about 1.75% w/w as determined by thermogravimetry.

In general, Form I of zolpidem hemitartrate has a moisture content of about 1.25% to 2.5% w/w as determined by Karl Fischer method.

The inventors also have developed a process for the preparation of the non-hygroscopic Form I of zolpidem hemitartrate. The process involves obtaining a solution of zolpidem in one or more alcoholic solvents; contacting the solution with L-(+)-tartaric acid to form a reaction mixture; adding an anti-solvent to the reaction mixture; and isolating the Form I of zolpidem hemitartrate from the mixture. The inventors also have developed pharmaceutical compositions that contain Form I of zolpidem hemitartrate in admixture with one or more solid or liquid pharmaceutical diluents, carriers, and/or excipients.

Alcoholic solvents include one or more of methanol, ethanol, isopropanol, and n-butanol. Examples of anti-solvents that may be added to precipitate out salt of zolpidem include ketones such as acetone, methyl isobutyl ketone, ethyl methyl ketone, diisobutyl ketone, and mixtures thereof.

Zolpidem base may be prepared by any of the methods known in the art. In particular, it may be prepared by the method described above.

In general, the solution of zolpidem base may be obtained by dissolving zolpidem in one or more solvents. The solution of zolpidem in a solvent can be obtained by dissolving, slurrying, stirring, or a combination thereof. The solution may be treated with animal charcoal before precipitation of the zolpidem hemitartrate.

In general, an anti-solvent may be added to induce precipitation of zolpidem hemitartrate from the reaction mixture. An anti-solvent is a solvent in which the salt of zolpidem is insoluble or sparingly soluble to the solvent in which salt of zolpidem is prepared. The precipitation may be spontaneous depending upon the solvents and the conditions used. The precipitation may also be facilitated by adding seeds of the desired polymorphic form. Alternatively, precipitation may also be induced by distilling off some solvent and/or reducing the temperature. For example, the desired polymorphic form made according to the latter method may be used as the seed in the former method at a subsequent time.

The product obtained may be further or additionally dried to achieve the desired moisture values. For example, the product may be further or additionally dried in a tray drier, dried under vacuum and/or in a Fluid Bed Drier.

For the purpose of this disclosure, a warm-blooded animal is a member of the animal kingdom possessed of a homeostatic mechanism and includes mammals and birds.

The present invention is further illustrated by the following examples which are provided merely to be exemplary of the invention and do not limit the scope of the invention. Certain modifications and equivalents will be apparent to those skilled in the art and are intended to be included within the scope of the present invention.

### Methods

### (a) X-Ray Powder Diffraction

X-ray powder diffraction patterns were recorded using the following instrument and parameters:
X-Ray Difractometer, Rigaku Coorperation, RU-H3R
Goniometer CN2155A3
X-Ray tube with Cu target anode
Divergence slits 1°, receiving slit 0.15 mm, Scatter slit 1°
Power: 40 KV, 50 mA
Scanning speed: 3 deg/min step: 0.02 deg
Wave length: 1.5406 A

### (b) Infrared Spectra

Infrared spectra were recorded using the following instrument and parameters: SCAN: 16 scans, 4.0 cm⁻¹
According to the USP 25, general test methods page 1920, infrared absorption spectrum by potassium bromide pellet method.

### (c) Differential Scanning Calorimetry

Differential scanning calorimetry plots were recorded using the following instrument and parameters:
DSC821 e, Mettler Toledo
Sample weight: 8-10 mg
Temperature range: 25-300° C.
Heating rate: 10° C./min
Nitrogen 20.0 mL/min
Number of holes in the crucible: 1
   (d) TGA analysis was carried out using Perkin Elmer Instrument
   (e) Moisture content determination was carried out by Karl-Fischer titration technique using Mettler instrument.

### EXAMPLE 1

### Preparation of Zolpidem

3-bromo-N,N-dimethyl-4-oxo-4-p-tolyl-butyramide (50 gm) was dissolved in methyl isobutyl ketone (350 ml) and stirred to get a solution. 2-amono-5-methylpyridine (18.1 gm) was added to this solution. The reaction mixture was heated to 82-85° C. and stirred at 82-85° C. for 18-20 hours. The reaction mixture was then cooled to 25° C. and the separated solids were filtered. The wet cake was washed with methyl isobutyl ketone (2×100 ml). The wet solid was suspended in de-ionized water (250 ml) and the pH was adjusted to 6.8-7.2 with aqueous sodium carbonate solution (10% w/v, 60 ml). The resultant mixture was stirred at room temperature for 30 minutes and filtered. The filtered solids were washed with de-ionized water (2×100 ml) and dried at 50° C. under reduced pressure to get zolpidem base.

### Yield: 12 g

### EXAMPLE 2

### Preparation of Zolpidem Hemitartrate

Zolpidem base (35 gm) was dissolved in methanol (140 ml) and 1.75 gm activated carbon was added to it. The resultant mass was stirred at room temperature for 15 minutes and then filtered through a celite bed. To the clear filtrate, a solution of L-(+)-tartaric acid (8.55 gm) dissolved in methanol (70 ml) was added under stirring at 45-50° C. Acetone (280 ml) was added to the reaction mass. The reaction mixture was seeded with pure zolpidem tartarate (0.2 gm) followed by cooling to −20 to −15° C. The resultant reaction mass was stirred at −20 to −15° C. for further 2 hours and separated solids were filtered. The wet cake was washed with acetone (2×70 ml). The cake was dried at 45 to 50° C. under reduced pressure for 6 to 8 hours to get pure zolpidem hemitartarate.

### Yield: 4.2 g (92.04%)

### EXAMPLE 3

### Preparation of Form I of Zolpidem Hemitartrate

Zolpidem base (30 gm) was dissolved in methanol (120 ml) and activated carbon (1.5 gm) was added to it. The resultant mass was stirred at room temperature for 15 minutes and then filtered through a celite bed and the bed was washed with methanol (2×30 ml). To the combined, clear filtrate, a solution of L-(+)-tartaric acid (7.2 gm) dissolved in methanol (60 ml) was added under stirring at 45-50° C. To the reaction mass, acetone (240 ml) was added. The reaction mixture was cooled to −20 to −15° C. The resultant reaction mass was stirred at −20 to −15° C. for further 2 hours and separated crystals were filtered. The cake was washed with acetone (2×55 ml). The cake was dried at 45 to 50° C. under reduced pressure to get Form I of zolpidem hemitartrate.

### Yield: 31.0 g

### Moisture Content: 2.33% w/w

TGA Analysis: Weight loss of 1.06% w/w
XRD pattern of Form I as depicted in FIG. I
DSC profile of Form I as depicted in FIG. II
IR Spectrum of Form I as depicted in FIG. III.

While the present invention has been described in terms of its specific embodiments, certain modifications and equivalents will be apparent to those skilled in the art and are intended to be included within the scope of the present invention. For example, the resulting zolpidem hemitartrate Form I can be used in pharmaceutical compositions with pharmaceutically acceptable carriers, excipients or diluents, as known by those of ordinary skill in the art, to prepare dosage forms that are suitable for administering to patients to treat the conditions described herein or otherwise known to be suitable for treatment with zolpidem.

We claim:

1. A process for the preparation of zolpidem of Formula I, 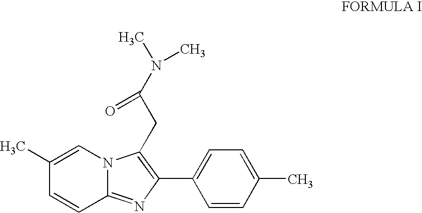
or a salt thereof, the process comprising:
a) condensing a bromo amide of Formula IV, 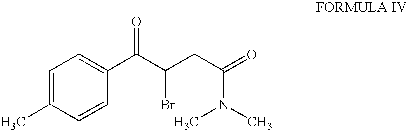
with 2-amino-5-methylpyridine in one or more polar aprotic solvents at a reaction temperature above 80° C.; and
b) isolating zolpidem of Formula I or a salt thereof, by the removal of the solvent.

2. The process of claim 1, wherein the solvent used in step a) has a boiling point above 80° C.

3. The process of claim 1, wherein the solvent comprises one or more of methyl isobutyl ketone, ethyl methyl ketone, diisobutyl ketone, 1,4-dioxane, sulpholane, dimethylformamide, dimethylacetamide, acetone, acetonitrile, N-methylpyrrolidone, and mixtures thereof.

4. The process of claim 3, wherein the solvent is methyl isobutyl ketone.

5. The process of claim 1, wherein the reaction temperature is from about 80° C. to about 120° C.

6. The process of claim 5, wherein the reaction temperature is from about 80° C. to about 100° C.

7. The process of claim 1, wherein removing the solvent comprises one or more of distillation, distillation under vacuum, evaporation, filtration, filtration under vacuum, decantation and centrifugation.

8. The process of claim 1, further comprising additional drying of the product obtained.

9. The process of claim 1, further comprising forming the product obtained into a finished dosage form.

10. The process of claim 1, wherein the salt is zolpidem hemitartrate.

11. The process of claim 10, wherein the zolpidem hemitartrate is prepared from zolpidem by treating it with L-(+)-tartaric acid.

12. A non-hygroscopic polymorphic Form I of zolpidem hemitartrate.

13. The Form I of zolpidem hemitartrate of claim 12 having characteristic X-ray diffraction peaks at two-theta values of 7.0, 7.8, 8.6, 8.9, 12.2, 15.6, 16.5, 17.3, 24.3 and 26.0.

14. The Form I of zolpidem hemitartrate of claim 12 having X-ray diffraction pattern of FIG. I.

15. The Form I of zolpidem hemitartrate of claim 12 having characteristic differential scanning calorimetry endothermic peaks at about 70° C., 109° C., 189° C. and 204° C.

16. The Form I of zolpidem hemitartrate of claim 15 having exothermic peaks at about 119° C. and 157° C.

17. The Form I of zolpidem hemitartrate of claim 12 having differential scanning calorimetry profile of FIG. II.

18. The Form I of zolpidem hemitartrate of claim 12 having characteristic thermogravimetric weight loss from about 1.0% w/w up to about 1.75% w/w.

19. The Form I of zolpidem hemitartrate of claim 12 having moisture content of about 1.25% w/w to 2.5% w/w as determined by Karl Fischer method.

20. The Form I of zolpidem hemitartrate of claim 12 having infrared spectrum of FIG. III.

21. A process for the preparation of Form I of zolpidem hemitartrate, the process comprising:
a) obtaining a solution of zolpidem base in one or more alcoholic solvents;
b) contacting the solution with L-(+)-tartaric acid;
c) adding an anti-solvent to the reaction mixture; and
d) isolating Form I of zolpidem hemitartrate from the mixture thereof.

22. The process of claim 21, wherein temperature of the reaction mixture is from about −50° C. to about 25° C.

23. The process of claim 21, wherein the alcoholic solvent comprises one or more of methanol, ethanol, isopropanol, n-butanol, and mixtures thereof.

24. The process of claim 23, wherein the alcoholic solvent is methanol.

25. The process of claim 21, wherein the anti-solvent comprises one or more of acetone, methyl isobutyl ketone, ethyl methyl ketone, diisobutyl ketone, and mixtures thereof.

26. The process of claim 25, wherein the anti-solvent is methyl isobutyl ketone.

27. A pharmaceutical composition comprising:
a therapeutically effective amount of Form I of zolpidem hemitartrate; and
one or more pharmaceutically acceptable carriers, excipients or diluents.

28. A method of treating anxiety, insomnia, sleep disorders, and convulsions in a warm-blooded animal, the method comprising providing a pharmaceutical composition to the warm-blooded animal, the pharmaceutical composition comprising Form I of zolpidem hemitartrate.
